(19) United States
(12) Patent Application Publication
(10) Pub. No.: US 2025/0009333 A1
(43) Pub. Date: Jan. 9, 2025

(54) FOCUSED ULTRASOUND APPARATUS FOR WOMEN

(71) Applicant: HIRONIC CO., LTD., Yongin-si (KR)

(72) Inventors: Woo Sung LEE, Namyangju-si (KR); Jung Ho PARK, Osan-si (KR); Eun Ju PARK, Uijeongbu-si (KR)

(21) Appl. No.: 18/764,865

(22) Filed: Jul. 5, 2024

(30) Foreign Application Priority Data
Jul. 7, 2023 (KR) 10-2023-0088131

(51) Int. Cl. A61B 8/12; A61B 8/00; A61B 8/08

(52) U.S. Cl. CPC A61B 8/12; A61B 8/085; A61B 8/4461

(57) ABSTRACT

A focused ultrasound apparatus for women is disclosed. According to one aspect of the present disclosure, the focused ultrasound apparatus for women is provided, including a handpiece, a cartridge which is detachably coupled to the handpiece and one side of the cartridge may be inserted into recipient's vagina, an ultrasound generation unit which is equipped on one side of the cartridge and generates a focused ultrasound, a rotation drive unit which rotates the cartridge which is equipped in and coupled to the handpiece around a longitudinal axis of the cartridge, a control unit which controls operations of the ultrasound generation unit and the rotation drive unit, and a procedure assistance unit which is movably coupled to the cartridge in order to enable an adjustment of an insertion depth of the cartridge inserted into a recipient's vagina.

### CROSS REFERENCE OF RELATED APPLICATION

This application claims the priority benefit of Korean Application No. 10-2023-0088131, filed Jul. 7, 2023, the entire content of which is incorporated herein for all purposes by this reference.

### BACKGROUND

### Field of the Invention

The present disclosure relates to a focused ultrasound apparatus for women.

### Description of the Related Art

Recently, the number of patients with gynecological diseases has been steadily increasing. The gynecological diseases may be broadly classified into a neoplastic disease, an inflammatory disease, a menstrual disorder, a sexually transmitted disease, and a sexual dysfunction, etc. More specifically, representative tumors include a cervical cancer and an ovarian cancer, etc., and a neoplastic disease includes a uterine fibroid. An inflammatory disease includes a cold leucorrhea, and menstrual disorders include an amenorrhea, a menstrual pain and an abnormal menstrual cycle, etc. Additionally, sexual dysfunctions include a sexual desire disorder, a sexual arousal disorder, an orgasm disorder, a pain disorder, and a vaginal contraction disorder, etc. Among these, the sexual dysfunction refers to a symptom of a difficulty or an inability to obtain a satisfaction through a sexual activity. Traditionally, a woman's sexual dysfunction has often been a natural disorder caused by childbirth or aging, but recently, the number of patients with the sexual dysfunction has been steadily increasing even among young people in their 20s and 30s.

There are various treatments for these gynecological diseases, which may be broadly divided into a pharmaceutical treatment and a medical device treatment. Among these, treatments that medical devices for treating these gynecological diseases are used refer to those that a heat therapy device, a moxibustion device, a sitz bath device, and a laser therapy device, etc. are used. However, the treatments that the heat therapy device, the moxibustion device, and the sitz bath device, etc. are used are not direct treatments, and, furthermore, are also known to have very little effect. In a case of a treatment using the laser therapy device, a strong pain and bleeding occur when performing a procedure, and a pain and side effect occur even after the procedure, causing a great inconvenience in daily life.

### SUMMARY

It is one of objects of the present disclosure to solve the above-mentioned problems. Various aspects of the present disclosure are directed to providing a focused ultrasound apparatus for women which, by rotating a cartridge equipped with an ultrasound generation unit that generates an ultrasound, irradiates the ultrasound into a recipient's vagina.

According to one aspect of the present disclosure, the focused ultrasound apparatus for women is provided, including a handpiece, a cartridge which is detachably coupled to the handpiece and one side of the cartridge may be inserted into recipient's vagina, an ultrasound generation unit which is equipped on one side of the cartridge and generates a focused ultrasound, a rotation drive unit which rotates the cartridge which is equipped in and coupled to the handpiece around a longitudinal axis of the cartridge, a control unit which controls operations of the ultrasound generation unit and the rotation drive unit, and a procedure assistance unit which is movably coupled to the cartridge in order to enable an adjustment of an insertion depth of the cartridge inserted into a recipient's vagina.

The rotation drive unit, concluding: a power generation portion equipped in the handpiece; and a rotation portion configured to rotate by being connected with the power generation portion, equipped at one end of the handpiece, wherein the cartridge, concluding: a coupling portion formed at an end of the cartridge, composed of a structure corresponding to the rotation portion and detachably coupled to the rotating portion.

The focused ultrasound apparatus, further concluding: a transmission unit configured to transmit a control signal and a power, disposed to correspond to the rotation portion and the coupling portion.

A detachment groove is formed in the rotation portion; and
a detachment portion is formed, engaging into the detachment groove in the rotation portion and supporting a combination of the cartridge and the handpiece, when the coupling portion is inserted into the rotation portion.

The focused ultrasound apparatus, further comprising: a motion detection unit configured to detect a motion of the handpiece, wherein the control unit receives a data on a motion of the handpiece from the motion detection unit and controls an operation of the ultrasound generation unit.

When an operation of the ultrasound generation unit starts, the control unit controls the rotation drive unit in order to rotate the cartridge.

In case that the handpiece does not operate or stops after the operation of the ultrasound generation unit has started, the control unit allows the operation of the ultrasound generation unit to stop.

The cartridge has a plurality of coupling grooves formed on outer peripheral surface of the cartridge along a longitudinal direction of the cartridge; and a coupling protrusion is formed on inner peripheral surface of the procedure assistance unit, engaging in and coupled to the coupling grooves.

The procedure assistance unit is allowed to be inserted into and coupled to an end of the handpiece.

### BRIEF DESCRIPTION OF DRAWINGS

FIG. 1 is a perspective view showing a focused ultrasound apparatus for women according to an embodiment of the present disclosure.

FIG. 2 is a state diagram showing a cartridge rotation of the focused ultrasound apparatus for women according to an embodiment of the present disclosure.

FIG. 3 is a diagram showing an internal structure of a handpiece of the focused ultrasound apparatus for women according to an embodiment of the present disclosure.

FIG. 4 is a diagram showing a rotation drive unit of the focused ultrasound apparatus for women according to an embodiment of the present invention.

FIG. 5 is a diagram showing a procedure assistance unit of the focused ultrasound apparatus for women according to an embodiment of the present disclosure.

FIG. 6 is a diagram showing a coupling state of the procedure assistance unit of the focused ultrasound apparatus for women according to an embodiment of the present disclosure.

### DETAILED DESCRIPTION

Since the present disclosure may be modified in various ways and may have various embodiments, specific embodiments will be illustrated in drawings and described in detail in detailed descriptions. However, this is not intended to limit the present disclosure to the specific embodiments, and should be understood to include all transformations, equivalents, and substitutes included in a spirit and technical scope of the present disclosure. In describing the present disclosure, if it is determined that detailed descriptions of related known technologies may obscure a gist of the present disclosure, the detailed descriptions will be omitted.

Terms such as first, second, etc. may be used to describe various components, but the components should not be limited by the terms. The above terms are used only for a purpose of distinguishing one component from another.

Terms used in the present application are only used to describe the specific embodiments and are not intended to limit the present disclosure. Singular expressions include plural expressions unless a context clearly dictates otherwise. In the present application, terms such as “comprise” or “have”, etc. are intended to designate a presence of a feature, a number, a step, an operation, a component, a part, or a combination thereof described in a specification, and it should be understood that this does not exclude in advance a presence or addition possibility of one or more other features, a number, a step, an operation, a component, a part, or a combination thereof.

In addition, coupling does not only refer to a case of direct physical contact among each component in a contact relationship among each component, but is also to be used as a concept that includes cases that other components are interposed among each component and components are respectively in contact with the other components.

Hereinafter, embodiments of a focused ultrasound apparatus (100) for women according to the present disclosure will be described in detail with reference to accompanying drawings. In addition, when explaining with reference to the accompanying drawings, identical or corresponding components will be assigned the same drawing numbers, and redundant explanations thereof will be omitted.

According to an embodiment of the present disclosure, the focused ultrasound apparatus (100) for women is provided, including a handpiece (110), a cartridge (120) which is detachably coupled to the handpiece (110) and one side of the cartridge may be inserted into a recipient's vagina, an ultrasound generation unit (130) which is equipped on one side of the cartridge (120) and generates a focused ultrasound, a rotation drive unit (140) which rotates the cartridge (120) which is equipped in and coupled to the handpiece (110) around a longitudinal axis of the cartridge (120), a control unit (150) which controls operations of the ultrasound generation unit (130) and the rotation drive unit (140), and a procedure assistance unit (160) which is movably coupled to the cartridge (120) in order to enable an adjustment of an insertion depth of the cartridge (120) inserted into a recipient's vagina.

According to the focused ultrasound apparatus (100) for women of the present embodiment, an ultrasound in all directions inside a recipient's vagina may be irradiated by rotating the cartridge (120) equipped with the ultrasound generation unit (130) that generates the ultrasound.

Additionally, in the present embodiment, a motion of the handpiece (110) may be detected through a motion detection unit (not shown) to be described later, through which, only when the cartridge (120) moves inside a recipient's vagina, a redundant irradiation to the same area is allowed to be prevented by operating the ultrasound generation unit (130). Furthermore, an ultrasound in a wide area is allowed to be irradiated by rotating the cartridge (120) according to an operation of the handpiece (110).

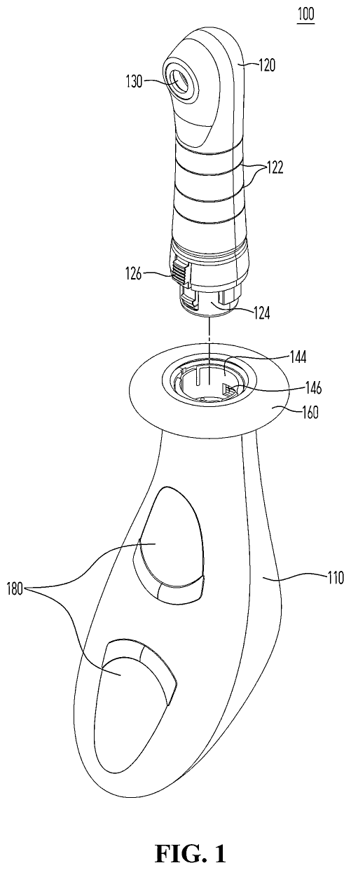

Hereinafter, each configuration of the focused ultrasound apparatus (100) for women according to the present embodiment will be described with reference to FIGS. 1,2,3,4,5, and 6.

First, FIG. 1 is a perspective view of the focused ultrasound apparatus (100) for women in the present embodiment, showing the handpiece (110) and the cartridge (120) in a separated state, and a coupling structure of a rotation portion (144) of the handpiece (110) and a coupling portion (124) of the cartridge (120) may be confirmed.

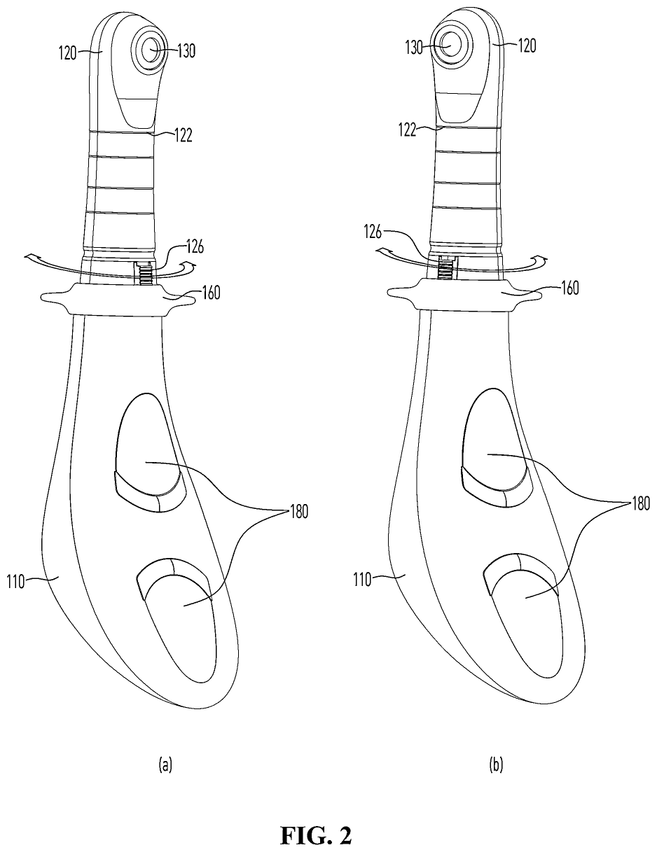

FIG. 2 shows that the cartridge (120) rotates around a longitudinal axis of the cartridge (120) in a state that the handpiece (110) and the cartridge (120) of the present embodiment are coupled, which rotation may occur by an operation of the rotation drive unit (140).

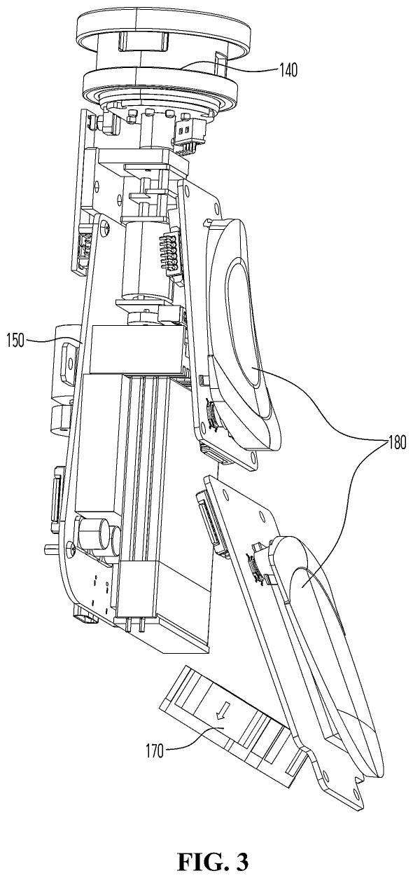

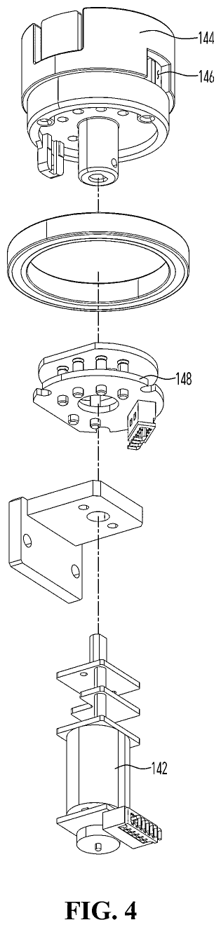

FIG. 3 shows an internal structure of the handpiece (110) of the present embodiment. An arrangement structure of the rotation drive unit (140), the control unit (150), a circulation fan (170), and an operation unit (180) may also be confirmed and FIG. 4 is an exploded perspective view of the rotation drive unit (140).

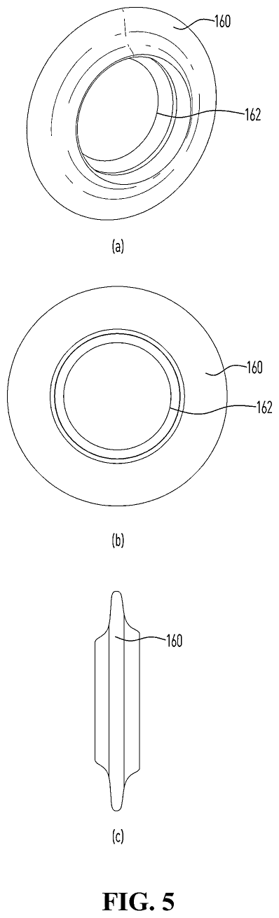

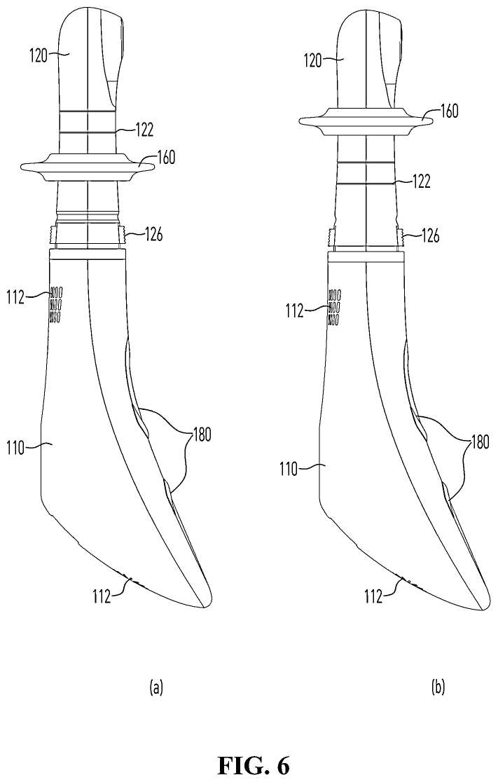

FIG. 5 shows a perspective view, a front view, and a side view of the procedure assistance unit (160). FIG. 6 is also a view showing the procedure assistance unit (160) coupled to the cartridge (120).

As shown in FIGS. 1, 2, and 6, the handpiece (110) may be held by an operator, and the operation unit (180) that controls an operation of the ultrasound generation unit (130) may be equipped on one side of the handpiece, and the cartridge (120) may be detachably coupled to one end of the handpiece.

Additionally, the handpiece (110) may be shaped so that the other end of the handpiece (110) is thicker than one end to which the cartridge (120) is coupled in order for an operator to easily withdraw the cartridge (120) after inserting the cartridge (120) into a recipient's vagina when performing a procedure.

As shown in FIG. 3, the rotation drive unit (140), the control unit (150), the operation unit (180), and the circulation fan (170) may be equipped inside the handpiece (110) and a power source which provides a power may also be disposed.

At this point, the circulation fan (170) circulates an air inside and outside the handpiece (110), so that a heat, which has been generated from the rotation drive unit (140), the control unit (150), and the power source inside the handpiece (110), may be discharged. As shown in FIG. 6, a ventilation hole (112) may be formed on an outer surface of the handpiece (110) in order to allow the air to be circulated.

One of these ventilation holes (112) may be disposed at a location adjacent to the circulation fan (170) and another of them may be disposed at a location spaced apart from the circulation fan (170), so that an air is allowed to be circulated overall inside the handpiece (110).

As shown in FIGS. 1, 2, and 6, the cartridge (120), which is detachably coupled to the handpiece (110), may be rotatably coupled 360 degrees by the rotation drive unit (140). Additionally, the cartridge (120) may be formed in a structure that one side of the cartridge (120) may be inserted into a recipient's vagina, and the ultrasound generation unit (130), which generates a focused ultrasound, may be equipped on the other side of the cartridge (120).

At this point, the ultrasound generation unit (130) may generate the focused ultrasound and may irradiate it outside the cartridge (120) through a window formed in the cartridge (120). When the ultrasound generation unit (130) irradiates the focused ultrasound in a state that the cartridge (120) is inserted into a recipient's vagina, thermal lesions may be formed on a recipient's vaginal skin tissue.

These thermal lesions may damage or irritate an endopelvic fascia, through which, a vaginal contractility may be improved.

In addition, while the handpiece (110) coupled to the cartridge (120) moves or the cartridge (120) rotates, the ultrasound generation unit (130) of the present embodiment operates, which may also prevent a redundant irradiation of the same area.

As shown in FIGS. 1, 3, and 4, the rotation drive unit (140), which is equipped in the handpiece (110), may rotate the cartridge (120) coupled to the handpiece (110) around a longitudinal axis of the cartridge (120). More specifically, the rotation drive unit (140) is equipped at a power generation portion (142) equipped in the handpiece (110) and at one end of the handpiece (110), and a rotation portion (144), which is connected with the power generation portion (142) and rotates, may be included.

At this point, the rotation portion (144) may be structured to be inserted into an inside of the hand piece (110) at one end of the handpiece (110), and as shown in FIG. 1, in this rotation portion (144), since a coupling portion (124) is formed at an end of the cartridge (120) and has a structure corresponding to the rotation portion (144), the coupling portion (124), which is detachably coupled to the rotation portion (144), may be inserted and coupled.

In addition, as shown in FIGS. 1 and 4, a detachment groove (146) may be formed in the rotation portion (144), and when the coupling portion (124) is inserted into the rotation portion (144) and engages in the detachment groove (146), a detachment portion (126) which supports a combination of the cartridge (120) and the handpiece (110) may be formed.

At this point, when one side of the detachment portion (126) is pressed in a state that the handpiece (110) is coupled to the cartridge (120), a part of one end of the cartridge (110), which has engaged in the detachment groove (146), is separated from the detachment groove (146) and, therefore, the combination of the handpiece (110) and the cartridge (120) may be released.

As shown in FIG. 4, since a transmission unit (148) is disposed to mutually correspond to the rotation portion (144) and the coupling portion (124), the transmission unit (148) may transmit a control signal and a power. This transmission unit (148) may also be coupled to the rotation portion (144) so that the control signal and the power may be transmitted even while the rotation portion (144) rotates.

As shown in FIG. 3, the control unit (150) is equipped inside the handpiece (110) and may control operations of the ultrasound generation unit (130) and the rotation drive unit (140). An operation of the ultrasonic generation unit (130) may also be controlled by receiving a data on a motion of the handpiece (110) from the motion detection unit (not shown) that detects the motion of the handpiece (110).

Since controlling the rotation drive unit (140) in order to rotate the cartridge (120) when an operation of the ultrasound generation unit (130) starts, this control unit (150) may prevent a focused ultrasound from being irradiated to the same location and may allow the focused ultrasound to be irradiated overall inside a recipient's vagina.

In addition, if a motion of the handpiece (110) does not occur or stops after the operation of the ultrasound generation unit (130) has begun, the control unit (150) may prevent the focused ultrasound from being irradiated to the same location by stopping the operation of the ultrasound generation unit (130).

For example, in the present embodiment, as the cartridge (120) is withdrawn while the ultrasound generation unit (130) is operated and, at the same time, the rotation drive unit (140) rotates the cartridge (120) in a state that the cartridge (120) is inserted into a recipient's vagina, spiral-shaped thermal lesions may be formed inside the recipient's vagina.

For example, the control unit (150) may control so that the ultrasound generation unit (130) may irradiate an ultrasound while rotating the cartridge (120) by operating the rotation drive unit (140). More specifically, whenever the rotation drive unit (140) rotates the cartridge (120) by 4.5 degrees, the control unit (150) may control the ultrasound generation unit (130) and the rotation drive unit (140) so that the ultrasound generation unit (130) irradiates an ultrasound.

Meanwhile, the motion detection unit (not shown) may detect a moving direction, a rotation, and an acceleration of the handpiece (110), and the control unit (150) may control an operation of the ultrasound generation unit (130) based on a data about a motion of the handpiece (110).

As shown in FIGS. 1, 2, 5 and 6, the procedure assistance unit (160) may be movably coupled to the cartridge (120) so that an insertion depth of the cartridge (120) inserted into a recipient's vagina may be adjusted.

More specifically, the cartridge (120) may have a plurality of coupling grooves (122) formed on an outer peripheral surface along a longitudinal direction of the cartridge (120), and a coupling protrusion (162), which is coupled by engaging in the coupling groove (122), may be formed on an inner peripheral surface of the procedure assistance unit (160). In addition, since the procedure assistance unit (160) is made of an elastic material, the procedure assistance unit (160) may be easily moved among a plurality of the coupling grooves (122).

As shown in FIG. 6, the procedure assistance unit (160) may be moved and coupled to a plurality of the coupling grooves (122), through which, an operator may easily recognize a length at which the cartridge (120) is inserted into a recipient's vagina.

In a process of using the focused ultrasound apparatus (100) for women in the present embodiment, before an operator inserts the cartridge (120) into a recipient's vagina, the procedure assistance unit (160) may be positioned in the coupling groove (122) formed in the cartridge (120) according to an ultrasound irradiation target position.

This procedure assistance unit (160) may assist so that an operator may recognize and adjust an insertion depth of the cartridge (120) without her or his visual confirmation. Furthermore, a recipient may use the focused ultrasound apparatus (100) for women according to the present embodiment, controlling an insertion depth of the cartridge (120) for herself or himself.

In addition, in a state that the cartridge (120) is inserted into a recipient's vagina, the procedure assistance unit (160) may cover an area around the recipient's vagina. Furthermore, a structure of this procedure assistance unit (160) may prevent a gel from flowing toward the handpiece (110), when the gel is used, so that an ultrasound irradiation efficiency may be increased and an insertion of the cartridge (120) be facilitated.

Meanwhile, as shown in FIG. 1, the procedure assistance unit (160) may be inserted into and coupled to an end of the handpiece (110). One side of the procedure assistance unit (160) may also have a wider inner diameter as shown in FIG. 5 (C) so that the procedure assistance unit (160) may be inserted into an end of the handpiece (110). The coupling protrusion (162) is in contact with one end of the handpiece (110), which position may be supported.

As mentioned above, the embodiments of the present disclosure have been described, however anyone skilled in the art may change, or modify the present disclosure in various way by annexing, changing, deleting or adding components, etc. without departing from a spirit of the present disclosure as set forth in the present patent claims, which will also be said to be included within a scope of rights of the present disclosure.

### DESCRIPTION OF SIGNS

100: Focused Ultrasound Apparatus for Women
110: Handpiece
112: Ventilation hole
120: Cartridge
122: Coupling Groove
124: Coupling Portion
126: Detachment Portion
130: Ultrasound Generation Unit
140: Rotation Drive Unit
142: Power Generation Portion
144: Rotation Portion
146: Detachment Groove
148: Transmission Unit
150: Control Unit
160: Procedure Assistance Unit
162: Coupling Protrusion
170: Circulation Fan
180: Operation Unit

What is claimed is:

1. A focused ultrasound apparatus for women,
the apparatus, comprising:
a handpiece;
a cartridge configured so that one side of the cartridge may be inserted into a recipient's vagina, detachably coupled to the handpiece;
an ultrasound generation unit configured to generate a focused ultrasound, equipped on one side of the cartridge;
a rotation drive unit configured to rotate the cartridge coupled to the handpiece around a longitudinal axis of the cartridge, equipped in the handpiece;
a control unit configured to control operations of the ultrasound generation unit and the rotation drive unit; and
a procedure assistance unit movably coupled to the cartridge in order to enable an adjustment of an insertion depth of the cartridge inserted into a recipient's vagina.

2. The focused ultrasound apparatus according to claim 1,
wherein the rotation drive unit, concluding:
a power generation portion equipped in the handpiece; and
a rotation portion configured to rotate by being connected with the power generation portion, equipped at one end of the handpiece,
wherein the cartridge, concluding:
a coupling portion formed at an end of the cartridge,
composed of a structure corresponding to the rotation portion and detachably coupled to the rotation portion.

3. The focused ultrasound apparatus according to claim 2, further concluding:
a transmission unit configured to transmit a control signal and a power, disposed to correspond to the rotation portion and the coupling portion.

4. The focused ultrasound apparatus according to claim 2,
wherein a detachment groove is formed in the rotation portion; and
a detachment portion is formed, engaging into the detachment groove in the rotation portion and supporting a combination of the cartridge and the handpiece, when the coupling portion is inserted into the rotation portion.

5. The focused ultrasound apparatus according to claim 1, further comprising:
a motion detection unit configured to detect a motion of the handpiece,
wherein the control unit receives a data on a motion of the handpiece from the motion detection unit and controls an operation of the ultrasound generation unit.

6. The focused ultrasound apparatus according to claim 5, wherein when an operation of the ultrasound generation unit starts, the control unit controls the rotation drive unit in order to rotate the cartridge.

7. The focused ultrasound apparatus according to claim 6, wherein in case that the handpiece does not operate or stops after the operation of the ultrasound generation unit has started, the control unit allows the operation of the ultrasound generation unit to stop.

8. The focused ultrasound apparatus according to claim 1,
wherein the cartridge has a plurality of coupling grooves formed on outer peripheral surface of the cartridge along a longitudinal direction of the cartridge; and
a coupling protrusion is formed on inner peripheral surface of the procedure assistance unit, engaging in and coupled to the coupling grooves.

9. The focused ultrasound apparatus according to claim 1, wherein the procedure assistance unit is allowed to be inserted into and coupled to an end of the handpiece.
